(19) United States
(12) Patent Application Publication
(10) Pub. No.: US 2013/0078642 A1
(43) Pub. Date: Mar. 28, 2013

(54) BIOMARKERS FOR LUNG NEUROENDOCRINE TUMORS

(71) Applicants: Maria Filippa Addis, Sassari (IT); Sergio Uzzau, Sassari (IT); Alessandro Tanca, Sassari (IT); Daniela Pagnozzi, Alghero (IT); Paolo Cossu Rocca, Sassari (IT); Giuseppe Fanciulli, Alghero (IT)

(72) Inventors: Maria Filippa Addis, Sassari (IT); Sergio Uzzau, Sassari (IT); Alessandro Tanca, Sassari (IT); Daniela Pagnozzi, Alghero (IT); Paolo Cossu Rocca, Sassari (IT); Giuseppe Fanciulli, Alghero (IT)

(73) Assignee: PORTO CONTE RICHERCHE S.R.L., Alghero (IT)

(21) Appl. No.: 13/701,420

(22) PCT Filed: Jun. 3, 2010
(86) PCT No.: PCT/EP2010/057785

§ 371 Date: Nov. 30, 2012

(51) Int. Cl. G01N 33/574

(52) U.S. Cl. CPC G01N 33/57484
USPC 435/6.12; 436/501; 435/7.92; 530/350; 435/189; 530/358

(57) ABSTRACT

The present invention regards a method for providing a diagnosis for small cell lung carcinoma and for typical carcinoid tumor by using a panel of protein biomarkers which are differentially expressed and a method for screening compounds.

### FIELD OF THE INVENTION

The present invention concerns a method for providing differential diagnosis among subgroups of lung neuroendocrine tumors. Specifically, the invention provides protein biomarkers which allow the discrimination of typical carcinoid tumor from small-cell lung carcinoma.

### STATE OF THE ART

Lung Neuroendocrine Tumors

Lung neuroendocrine tumors (lung NETs) enclose a spectrum of neoplastic lesions arising from neuroendocrine cells of the pulmonary epithelium, accounting for about 20% of all pulmonary cancers. They are currently classified into four subgroups: typical carcinoid tumor (TC), atypical carcinoid tumor (AC), large-cell neuroendocrine carcinoma (LCNEC), and small-cell lung carcinoma (SCLC) (1).

TCs are considered as low-grade malignancies with favorable prognosis which may benefit from complete surgical resection, whereas SCLCs show a highly aggressive behaviour and are usually treated with chemotherapy alone. Therefore, an accurate differential diagnosis is mandatory for a correct choice of therapy.

The frequent mistakes in differential diagnosis between TC and SCLC, often dependent on the small size of the samples obtained from bronchoscopy or CT-assisted biopsies, and the morphologic overlapping between these variants, have been described as one of the most important “pitfalls” in the management of lung cancers (2).

Up to now, in combination with the morphological examination, a large number of markers have been described as correlated to neoplastic diseases, but the need is increasingly felt for a differential diagnostic method for distinguishing between the lung neuroendocrine diseases. Several proteins, such as Ki-67, chromogranin A (CgA), neuron-specific enolase, serotonin, synaptophysin, and adrenocorticotrophic hormone, are used by pathologists for establishing a differential diagnosis. However, a differential marker with a satisfactory degree of sensitivity and specificity still needs to be found.

Candidate Biomarkers

In view of this, a differential proteomics study was conducted, exploiting a recently optimized method for extracting proteins from formalin-fixed, paraffin-embedded tissues (3). The analysis led to the identification of 35 differentially expressed proteins, which can be considered as candidate biomarkers for the differential diagnosis of lung NETs (see below).

A correlation between the expression of the greater part of the identified potential biomarkers and the development (or the diagnosis) of various types of cancer (different from lung NETs) has been previously demonstrated. Several variants of arginine/serine rich splicing factors, were identified as receptors for lung colonization of cancer cells (4). Among oxidoreductases, peroxiredoxins were seen to be expressed in several normal and tumoral tissues; in particular, type 6 is typical of lung (both healthy and diseased) (5); superoxide dismutase isoforms are present at heterogeneous ratios in cancer tissues, mostly in the lung, as a consequence of its constant exposition to oxygen.

Other proteins, more consistent with neuroendocrine differentiation, such as transthyretin and protein CutA, would be very valuable markers for distinguishing between NETs. Transthyretin plays a crucial role in senile systemic amyloidosis development; conversely, little is known about protein CutA, apart from its role in the acetylcholinesterase secretion pathway (6). Overexpression of various isoforms of heterogeneous nuclear ribonucleoproteins was demonstrated in different tumors (7). HnRNP A1 overexpression has been reported only in colon and lung cancer by means of quantitative gene expression analyses. Stathmin, also known as oncoprotein-18 (Op18), is an ubiquitous, highly conserved cytosolic protein which is primarily involved in regulation of microtubule dynamics, in relation to multisite phosphorylation of its serine residues. Upregulation of this protein was described in several neoplasms; its overexpression was demonstrated in neuroblastomas, high grade lymphomas and acute leukemias, and further identified in malignant epithelial neoplasms, such as ovarian, prostatic, breast, liver, and lung cancer (8).

It is therefore the object of the present invention the potential use of diagnostic markers (alone or in combination) for distinguishing between TC and SCLC, in order to allow a rapid and precise diagnosis of the malignancy and allow a correct choice of therapy.

### SUMMARY OF THE INVENTION

The present invention provides a method that allows a diagnosis for small cell lung carcinoma (SCLC) comprising the steps of:
a. Contacting a biological sample with reagents that allow the extraction of the protein biomarkers selected from the group consisting of: Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1A chain, Tubulin beta chain;
b. Determining whether the protein biomarkers are differentially expressed in the sample.

The present invention also provides a method that allows a diagnosis for typical carcinoid tumor (TC) comprising the steps of:
a. Contacting a biological sample with reagents that allow the extraction of the protein biomarkers selected from the group consisting of: Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin;
b. Determining whether the protein markers are differentially expressed in the sample.

According to a further aspect the invention provides the use of one or more protein biomarkers selected from group consisting of: Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1A chain, Tubulin beta chain, as biomarkers for small cell lung carcinoma.

According to a still further aspect, the invention provides the use of one or more protein biomarkers selected from group consisting of: Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin as biomarkers for typical carcinoid tumor.

A further object of the invention concerns a method of in vitro screening for a compound for treating small cell lung cancer comprising the steps of:
a. Contacting a polypeptide which encodes Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1 A chain, Tubulin beta chainwith a test compound;
b. Detecting the biological activity of the polypeptide of step a.

A still further object of the invention concerns a method of in vitro screening for a compound for treating typical carcinoid tumor comprising the steps of:
a. Contacting a polypeptide which encodes Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin with a test compound;
b. Detecting the biological activity of the polypeptide of step a.

The present invention also provides a method for providing a differential diagnosis between small cell lung carcinoma (SCLC) and typical carcinoid tumor (TC) by an antibody-based technique comprising the steps of,
a. Contacting a biological sample with an antibody against one of the following antigens: Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1A chain, Tubulin beta chain, Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin;
b. Detecting immunoreactivity.

In a preferred aspect said antibody-based technique is selected from the group consisting of: immunohistochemistry, immunofluorescence and ELISA.

### BRIEF DESCRIPTION OF THE DRAWINGS

The characteristics and advantages of the present invention will be apparent from the detailed description reported below, from the Examples given for illustrative and non-limiting purposes, and from the annexed Figures, wherein:

FIG. 1. shows Venn diagrams illustrating the distribution of the proteins identified among three samples of the same tumor subtype. A total of 102 and 79 proteins were common to all TC and SCLC samples, respectively, and can be considered typical of the tumor subclass. Combining these data, 11 proteins were detected exclusively in all TC samples, whereas only 3 proteins were detected exclusively in all SCLC samples.

FIG. 2. describes the results obtained with Immunohistochemical analyses.

FIG. 2A: immunohistochemistry for stathmin on SCLC showing a diffuse pattern of cytoplasmic staining (magnification 400×).

FIG. 2B: negative immunostaining for stathmin on TC neoplastic cells with sparse, intratumoral immunoreactive sustentacular cells as positive internal control (magnification 400×).

FIG. 2C: immunohistochemistry for hnRNP A1 on TC showing a diffuse pattern of nuclear staining (magnification 400×).

FIG. 2D: nuclear immunoreactivity for hnRNP A1 is also recognizable on normal bronchiolar cells of peritumoral lung parenchyma (magnification 400×).

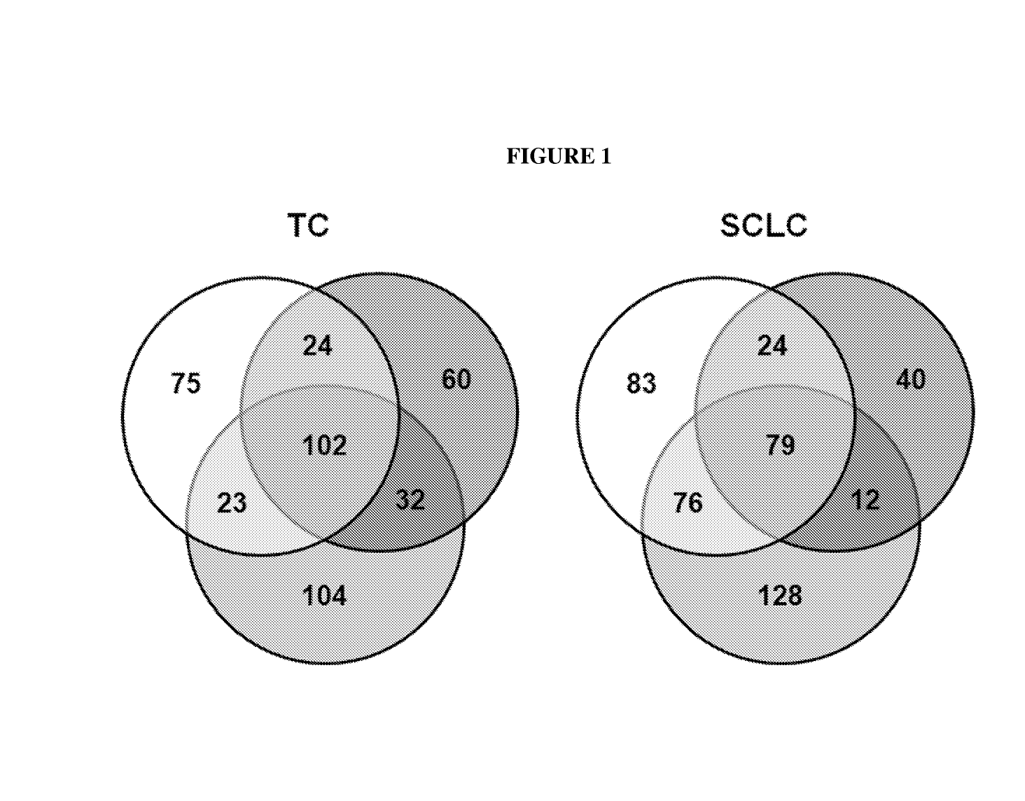

### DETAILED DESCRIPTION OF THE INVENTION

The present invention provides a method that allows a diagnosis for small cell lung carcinoma (SCLC) comprising the steps of:
a. Contacting a biological sample with reagents that allow the extraction of the protein biomarkers selected from the group consisting of: Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1A chain, Tubulin beta chain;
b. Determining whether the protein biomarkers are differentially expressed in the sample.

The present invention also provides a method that allows a diagnosis for typical carcinoid tumor (TC) comprising the steps of:
a. Contacting a biological sample with reagents that allow the extraction of the protein biomarkers selected from the group consisting of: Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin and
b. Determining whether the protein markers are differentially expressed in the sample.

The methods of the present invention have the advantages of allowing a differential diagnosis of TC and SCLC with a potential high degree of sensitivity and specificity and, in turn, a significant improvement in diagnostic efficiency. In the method provided by the present invention biological samples comprising a lung cell, fresh or frozen tissue samples or preferably formalin-fixed, paraffin embedded (FFPE) samples may be used.

Up to now it has been seen that there is a great difficulty in recovery of fresh and frozen tissues of neoplastic specimens. Tissue specimens which are not properly stored also risk degradation and therefore the proteomic analysis results very often impaired. One of the advantages of the present inventive method of providing a diagnosis of TC and SCLC, is that the biological samples may also be in the form of formalin-fixed, paraffin embedded (FFPE) samples, allowing a more efficient sample storage, the possibility of creating tissue banks which include valuable clinical information associated to each specimen and a more efficient long-distance sample exchange.

The differential expression of the method provided is determined by one of:
a. Detecting mRNA which encodes for the protein biomarker;
b. Detecting the protein encoded by the protein biomarker;
c. Detecting the biological activity of the protein biomarker.

The present invention also provides a method that allows a differential diagnosis between small cell lung carcinoma (SCLC) and typical carcinoid tumor (TC) by an antibody-based technique comprising the steps of,
a. Contacting a biological sample with an antibody against one of the following antigens: Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1 A chain, Tubulin beta chain, Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin;
b. Detecting immunoreactivity.

In a preferred aspect said antibody-based technique is selected from the group consisting of: immunohistochemistry, immunofluorescence and ELISA.

Preliminary immunohistochemical evaluation of stathmin differential expression between TC and SCLC surprisingly identifies this protein as a SCLC protein biomarker.

A further aspect of the present invention is a protein biomarker selected from the group consisting of: Elongation factor 1-alpha 1, Histone H1.2, Histone H1.5,

Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1A chain, Tubulin beta chain, Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin as a diagnostic reagent.

According to a further aspect the invention provides the use of one or more protein biomarkers selected from group consisting of: Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1 A chain, Tubulin beta chain as biomarkers for small cell lung carcinoma.

According to a still further aspect, the invention provides the use of one or more protein biomarkers selected from group consisting of: Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin as biomarkers for typical carcinoid tumor.

A further object of the invention concerns a method of in vitro screening for a compound for treating small cell lung cancer comprising the steps of:
a. Contacting a polypeptide which encodes Elongation factor 1-alpha 1, Histone H1.2, Histone H1.5, Histone H2A type 1-D, Histone H3.1t, Histone H4, Heterogeneous nuclear ribonucleoproteins Cl /C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1A chain, Tubulin beta chain factor with a test compound;
b. Detecting the biological activity of the polypeptide of step a.

A still further object of the invention concerns a method of in vitro screening for a compound for treating typical carcinoid tumor comprising the steps of:
a. Contacting a polypeptide which encodes Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin with a test compound;
b. Detecting the biological activity of the polypeptide of step a.

### EXAMPLES

### Example 1

### Preparation of Protein Extracts

Tissue Samples

Paraffin blocks from surgical specimens of 8 NETs, comprehensive of 4 TCs and 4 SCLCs were retrieved from the archives stored in the Departments of Pathology at the University Hospitals of Sassari and Verona, Italy. Four patients were male and 4 were female. Age of patients ranged from 52 to 73 years (mean: 57). Age of selected paraffin blocks ranged from 4 to 60 months (mean: 25). Hematoxylin and eosin stains were critically reviewed and the tumors were classified according to the WHO 2004 classification of neuroendocrine neoplasm of the lung.

Protein Extraction and Protein Quantification

Protein extraction from FFPE tissues was performed as reported previously (3). Microtome sections (10 μm thick, 80 mm² wide) were cut from FFPE tissue blocks and deparaffinized by incubation in xylene, rehydrated with a graded series of ethanol, immersed at a 20% w/v ratio in extraction buffer (EB), and subjected to high-temperature extraction. Protein extracts were clarified for 15 min at 12,000×g at 4° C., quantified by EZQ Protein quantification kit (Molecular Probes, Eugene, Oreg.), and stored at −80° C. until needed.

### Example 2

### Electrophoresis of Protein Extracts

Sodium Dodecyl Sulphate-PolyAcrylamide Gel Electrophoresis (SDS-PAGE)

Six human bioptic FFPE samples, three diagnosed as TCs, and three as SCLCs, were subjected to the protein extraction procedure described in Example 1.

Two identical aliquots of 20 micrograms of each protein were subjected and separated by SDS-PAGE (Laemmli), and gels were stained with Coomassie Brilliant blue G-250.

Gel images were digitalized with an ImageScanner III (GE Healthcare, Little Chalfont, UK).

### Example 3

### Liquid Chromatography-Mass Spectrometry: GeLC-MS/MS Analysis

In-Gel Trypsin Digestion

SDS-PAGE gels were then cut according to either the Visible Band (VB) method or the Whole lane (WL) method. Bands were destained by repetitive washings with 50 mM NH₄HCO₃, pH 8.0, and acetonitrile. Samples were reduced and carbamidomethylated in 50 mM NH₄HCO₃ buffer, pH 8.0, first with 10 mM DTT at 56° C., and then with 55 mM iodoacetamide at RT in the dark. Tryptic digestion of the alkylated samples was performed at 37° C. overnight using an average amount of 80 ng of trypsin per gel slice. For the VB method, the 13 clearly visible bands are excised, while in the case of the WL method, fractionation of the whole lane into 38 homogeneous slices, independently from band intensities is performed. In a previous work (4), a GeLC-MS/MS investigation of FFPE skeletal muscle tissue extracts was performed by cutting only bands which were visible after colloidal Coomassie staining. However, MS identification of formalin-fixed electrophoresed proteins was also achievable upon analysis of gel regions which did not show a visible signal. This evidence led us to apply GeLC-MS/MS also to gel slices cut from the whole electrophoresis lane in order to quantitatively and qualitatively improve proteomic coverage. Thus, a total of 306 gel portions were in situ digested with trypsin, extracted and analyzed by LC-MS/MS; finally, MS identification data were processed, in order to obtain a complete proteomic profile for each probed extract.

LC-MS/MS

LC-MS/MS analyses of tryptic digests were performed on a Q-TOF hybrid mass spectrometer equipped with a nano lock Z-spray source, and coupled on-line with a capillary chromatography system CapLC (Waters, Manchester, UK). After loading, the peptide mixture (6 μL) was first concentrated and washed at 20 μL/min onto a reverse-phase pre-column (Symmetry 300, C18, 5 μm, NanoEase, Waters) using 0.2% formic acid as eluent. The sample was then fractionated onto a C18 reverse-phase capillary column (Nanoflow column 5 μm Biosphere C18, 75 μm×200 mm, Nanoseparations) at a flow rate of 250 nL/min, using a linear gradient of eluent B (0.2% formic acid in 95% acetonitrile) in A (0.2% formic acid in 5% acetonitrile) from 7 to 50% in 40 min. Mass spectrometer was set up in a data-dependent MS/MS mode where a full scan spectrum (m/z acquisition range from 400 to 1600 Da/e) was followed by a tandem mass spectrum (m/z acquisition range from 100 to 2000 Da/e). Peptide ions were selected as the three most intense peaks of the previous scan. A suitable collision energy was applied depending on the mass and charge of the precursor ion. Argon was used as the collision gas. ProteinLynx software, provided by the manufacturers, was used to analyze raw MS and MS/MS spectra and to generate a peak list which was introduced in the in-house Mascot MS/MS ion search software (Version 2.2, Matrix Science, Boston, Mass.) for protein identification. Search parameters were as follows: peptide tolerance 30 ppm, MS/MS tolerance 0,4 Da, charge state +2 and +3, enzyme trypsin, allowing 1 missed cleavage.

Data Analysis

Total peptide hits (TPH) were used as a parameter for estimating and comparing protein abundance between samples in GeLC-MS/MS analyses. TPH value for each protein was calculated by summing the “queries matched” number (as indicated by Mascot software) of all gel bands arising from a particular sample (or sample class). Unique peptides (UP) and sequence coverage (SC) values reported in all tables have to be intended as the best value obtained in a single LC-MS/MS run. Only proteins which reported at least one peptide ranked by Mascot with a value equal to 1 were included. Queries matched to more than a protein hit (among protein hits obtained from a single LC-MS/MS run) were counted for each of the proteins. Skin keratins were excluded from the final protein list. Gene Ontology (GO) assignments were carried out using DAVID software (9).

Statistical analysis was carried out using a Kruskal-Wallis H test, which is a nonparametric version of one-way analysis of variance.

Considering k samples of sizes N1, N2, . . . , Nk, with N=sumNi, ranking all the data, regardless to the k samples, and indicating with R1, R2, . . . , Rk the sums of is the ranks for the k samples, the following statistic is defined:

H={12/N(N+1)}*sum {j=1 to k} (Rj\hat{0}2/Nj)−3(N+1).

Results

Overall GeLC-MS/MS results for each sample were generated by merging the proteomic information from the VB and WL replicates, while data concerning the two compared tumor types were obtained by combining profiles of single samples into classes.

Table 1, summarizes the number proteins detected for TC and SCLC. A total of 420 and 442 distinct proteins were detected, respectively. In total, a mean of almost 240 distinct proteins per sample were detected, whereas the TPH value per sample was around 5000.

TABLE 1
Summary of GeLC-MS/MS data, according to sample classes

| | Unique | THP: Total |
|---|---|---|
| Sample | proteins | Protein Hits |
| TC1 | 224 | 5624 |
| TC2 | 218 | 5146 |
| TC3 | 261 | 4981 |
| mean TC | 234 | 5250 |
| total TC | 420 | 15751 |
| SCLC1 | 262 | 5685 |
| SCLC2 | 155 | 4709 |
| SCLC3 | 295 | 3815 |
| mean SCLC | 237 | 4736 |
| total SCLC | 442 | 14209 |
| mean NET | 236 | 4993 |
| total NET | 616 | 29960 |

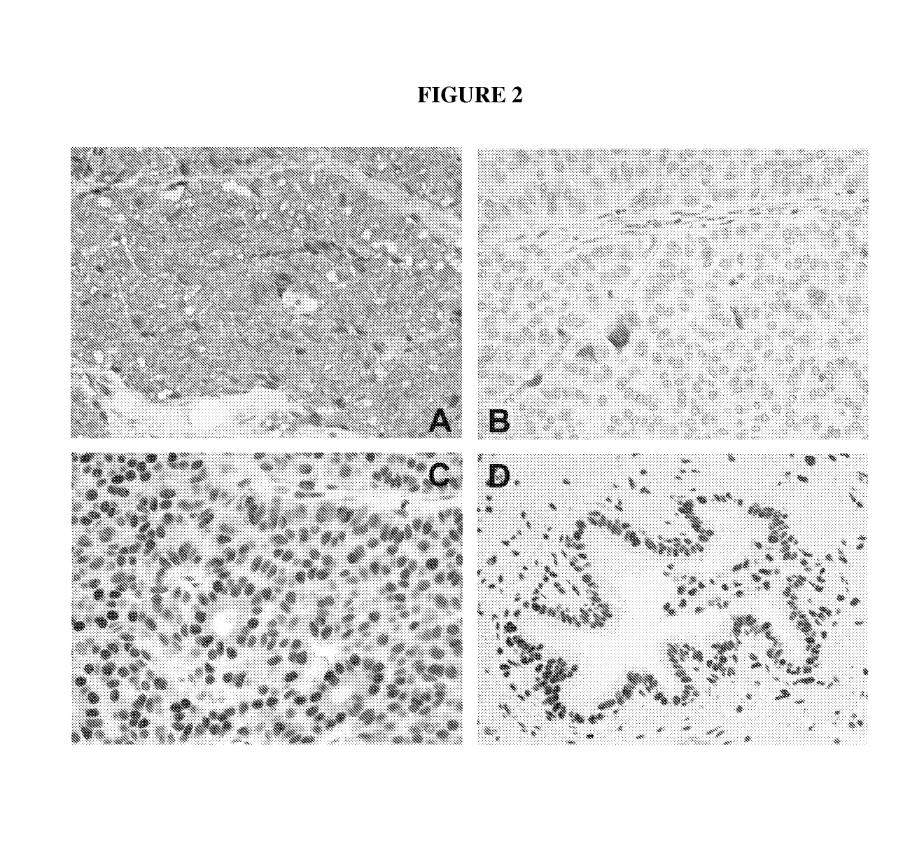

Sharing and intersection plots for protein identifications among samples of the same tumor subgroup are shown in FIG. 2. A total of 102 and 79 proteins were common to all TC and SCLC samples, respectively, and can be considered typical of the tumor subclass. Combining these data, 11 proteins were detected exclusively in all TC samples, whereas only 3 proteins were detected exclusively in all SCLC samples.

### Example 4

### Candidate Biomarker Identification by Statistical Analysis

A non-parametric Kruskal-Wallis test was performed on TPH data with the purpose of identifying a set of statistically-supported, robust, and dependable differentially expressed candidate biomarkers. The task of this statistical “filter” was to select is proteins which were homogeneously abundant among the samples of a disease class, and absent (or evenly under-expressed) within the other subgroup. Proteins falling into a 95% confidence interval are displayed in Table 2.

For the purposes of the present invention, each protein biomarker has a Uniprot/Swissprot accession number and a corresponding SEQ ID NO., as indicated in Table 2.

TABLE 2
List of proteins differentially expressed in TC and SCLC samples.

| Uniprot- | | | | | |
|---|---|---|---|---|---|
| Swiss- | | TC | SCLC | Candi- | |
| prot | | total | total | date | |
| Ac- | | pep- | pep- | bio- | |
| cession | | tide | tide | marker | SEQ ID |
| number | Protein name | hits | hits | for | NO. |
| O60888 | Protein CutA | 7 | 0 | TC | SEQ ID |
| | Superoxide dismutase | | | | NO. 1 |
| P00441 | (Cu—Zn) | 9 | 0 | TC | SEQ ID |
| | | | | | NO. 2 |
| P02042 | Hemoglobin subunit | 929 | 142 | TC | SEQ ID |
| | delta | | | | NO. 3 |
| P02766 | Transthyretin | 6 | 0 | TC | SEQ ID |
| | | | | | NO. 4 |
| P02794 | Ferritin heavy chain | 7 | 0 | TC | SEQ ID |
| | | | | | NO. 5 |
| P05109 | Protein S100-A8 | 15 | 1 | TC | SEQ ID |
| | | | | | NO. 6 |
| P07437 | Tubulin beta chain | 14 | 155 | SCLC | SEQ ID |
| | | | | | NO. 7 |
| P07910 | Heterogeneous nuclear | 13 | 29 | SCLC | SEQ ID |
| | ribonucleoproteins | | | | NO. 8 |
| | C1/C2 | | | | |
| P07988 | Pulmonary surfactant- | 5 | 0 | TC | SEQ ID |
| | associated protein B | | | | NO. 9 |
| P08294 | Extracellular | 8 | 0 | TC | SEQ ID |
| | superoxide | | | | NO. 10 |
| | dismutase (Cu—Zn) | | | | |
| P08758 | Annexin A5 | 49 | 17 | TC | SEQ ID |
| | Heterogeneous nuclear | | | | NO. 11 |
| P09651 | ribonucleoprotein A1 | 0 | 27 | SCLC | SEQ ID |
| | | | | | NO. 12 |
| P10645 | Chromogranin-A | 66 | 0 | TC | SEQ ID |
| | | | | | NO. 13 |
| P16401 | Histone H1.5 | 0 | 39 | SCLC | SEQ ID |
| | | | | | NO. 14 |
| P16403 | Histone H1.2 | 18 | 135 | SCLC | SEQ ID |
| | | | | | NO. 15 |
| P16949 | Stathmin | 1 | 34 | SCLC | SEQ ID |
| | | | | | NO. 16 |
| P18669 | Phosphoglycerate | 4 | 0 | TC | SEQ ID |
| | mutase 1 | | | | NO. 17 |
| P20671 | Histone H2A type 1-D | 138 | 859 | SCLC | SEQ ID |
| | | | | | NO. 18 |
| P30041 | Peroxiredoxin-6 | 19 | 3 | TC | SEQ ID |
| | | | | | NO. 19 |
| P30044 | Peroxiredoxin-5, | 6 | 0 | TC | SEQ ID |
| | mitochondrial | | | | NO. 20 |
| P37802 | Transgelin-2 | 46 | 22 | TC | SEQ ID |
| | | | | | NO. 21 |
| P61978 | Heterogeneous nuclear | 6 | 24 | SCLC | SEQ ID |
| | ribonucleoprotein K | | | | NO. 22 |
| P62701 | 40S ribosomal protein | 1 | 7 | SCLC | SEQ ID |
| | S4, X isoform | | | | NO. 23 |
| P62750 | 60S ribosomal protein | 1 | 13 | SCLC | SEQ ID |
| | L23a | | | | NO. 24 |
| P62805 | Histone H4 | 725 | 1857 | SCLC | SEQ ID |
| | | | | | NO.25 |
| P68104 | Elongation factor | 4 | 40 | SCLC | SEQ ID |
| | 1-alpha 1 | | | | NO.26 |
| P68871 | Hemoglobin subunit | 3459 | 1234 | TC | SEQ ID |
| | beta | | | | NO. 27 |
| P69905 | Hemoglobin subunit | 2580 | 854 | TC | SEQ ID |
| | alpha | | | | NO. 28 |
| | 60S ribosomal protein | | | | |
| Q07020 | L18 | 2 | 12 | SCLC | SEQ ID |
| | | | | | NO. 29 |
| Q13242 | Splicing factor, | 0 | 9 | SCLC | SEQ ID |
| | arginine/senne-rich 9 | | | | NO. 30 |
| Q16695 | Histone H3.1t | 104 | 404 | SCLC | SEQ ID |
| | | | | | NO. 31 |
| Q5JNZ5 | Putative 40S ribosomal | 1 | 9 | SCLC | SEQ ID |
| | protein S26-like 1 | | | | NO. 32 |
| Q71U36 | Tubulin alpha-1A chain | 37 | 223 | SCLC | SEQ ID |
| | | | | | NO. 33 |
| Q8IWL2 | Pulmonary surfactant- | 15 | 0 | TC | SEQ ID |
| | associated protein A1 | | | | NO. 34 |
| Q96IU4 | Abhydrolase domain- | 5 | 0 | TC | SEQ ID |
| | containing protein 14B | | | | NO. 35 |

Among ubiquitous proteins (globins, for instance), and well-known neuroendocrine markers (such as CgA), an interesting group of proteins was found, comprising superoxide dismutase, protein Cut A, peroxiredoxin-5, and protein S100-A8, which were more significantly expressed in TCs; on the other hand, hnRNP A1, splicing factor arginine/serine-rich 9, and stathmin were overexpressed with high statistical significance in SCLCs.

### Example 5

### Immunohistochemistry

Immunostaining with specific antibodies was performed as a preliminary evaluation of the differential diagnostic potential of two candidate biomarkers; specifically, the expression of stathmin and hnRNP A1 was analyzed in both TC and SCLC series involved in our study.

Stathmin expression was recognizable in all the SCLC variants, showing a diffuse pattern of cytoplasmic immunostaining with moderate to strong intensity (FIG. 2A), whereas TCs were consistently negative, with only rare, weakly immunoreactive neoplastic cells, and sparse, moderately stained intratumoral sustentacular cells (FIG. 2B). No immunostaining was appreciable in non-neoplastic adjacent pulmonary parenchyma.

Conversely, hnRNP Al expression was detected in both TC and SCLC samples, with diffuse nuclear immunostaining and strong intensity (FIG. 2C). Nuclear staining of moderate to strong intensity was also recognizable in peritumoral bronchiolar and alveolar cells (FIG. 2D).

Immunohistochemical evaluation of stathmin differential expression between TC and SCLC surprisingly shows strong and diffuse immunostaining for stathmin, specifically detected only in SCLC variants, identifying this protein as a suitable tumor marker.

### Example 6

### Immunofluorescence

An exemplary application of the present invention is the use of immunofluorescent methods in order to perform differential diagnosis of lung NETs. Dye-coupled antibodies can be used, in order to detect the presence of one or a combination of the abovementioned biomarkers in tissue sections, by means of fluorescence microscopy or confocal microscopy.

For instance, a human lung tissue section, coming from a FFPE surgical biopsy block, is treated with a panel of primary antibodies against antigens comprising stathmin, hnRNP C1/C2, and Splicing factor, arginine/serine-rich 9, then incubated with a fluorescent-labeled secondary antibody. Finally, the tissue slide is analyzed in fluorescence microscopy, allowing a specific and sensitive diagnosis of SCLC.

### Example 7

### Real Time PCR

A further example of an application of the present invention is the use of PCR-based methods, allowing the detection of mRNA encoding for polypeptides listed in Table 2. In particular, specific TaqMan probes are designed and properly combined in order to perform a highly sensitive differential diagnosis of lung NETs. For instance, RNA is extracted from lung tumor tissue samples, following standard procedures; then a Real Time PCR analysis is performed, using a panel of six TaqMan probes, specifically matching to the RNA transcripts of stathmin, tubulin alpha-1A chain, chromogranin-A, annexin A5, protein CutA, and histone H3. Such assay specifically detects and quantifies the expression of marker transcripts, allowing a diagnostic response of ten lung NETs cases in only one analysis.

### Example 8

### ELISA

A further exemplary use of the present invention is an enzyme-linked immunosorbent assay, aimed to detect the presence of one or a combination of said biomarkers in peripheral blood. The assay exploits specific antibodies directed against a panel of the selected antigens, able to give a quantitative measure of the protein in a patients’ serum/plasma, thus allowing a sensitive, rapid, non-invasive, and cost-effective diagnosis of lung neuroendocrine tumors.

For instance, blood samples are collected from three lung tumor patients and three healthy controls; serum/plasma are obtained according to standard procedures. An ELISA sandwich system is designed, comprising a 96-well assay plate, in which twelve different specific antibodies, directed against the same number of markers (such as Chromogranin-A, Histone H1.5, Histone H1.2, Stathmin, Phosphoglycerate mutase 1, Histone H2A type 1-D, Peroxiredoxin-6, Peroxiredoxin-5 mitochondrial, Transgelin-2, Heterogeneous nuclear ribonucleoprotein K, 40S ribosomal protein S4, X isoform, 60S ribosomal protein L23a), are adsorbed. Two-hundreds microliters of diluted serum/plasma are dispensed in each well and incubated at RT; then wells are washed, and incubated with a second specific (primary) antibody, followed by a secondary labeled antibody, directed against the second primary antibody. Finally, absorbance is measured by means of a spectrophotometer, and antigens are quantified in both samples and controls, in order to perform a sensitive and rapid diagnosis.

The novel methods for providing a diagnosis for small cell lung carcinoma (SCLC) and typical carcinoid (TC) according to the present invention and described in the Examples surprisingly show an improved diagnostic efficacy and an accurate differential diagnosis allowing a correct and precise therapeutic choice. Interestingly such methods can be used on samples extracted from formalin-fixed, paraffin embedded (FFPE) tissues, allowing a more effective biomarker analysis. From the above description and the above-noted Examples, the advantages attained by the methods described and obtained according to the present invention are apparent.

### REFERENCES

1. Gustafsson B I, Kidd M, Chan A, Malfertheiner M V, Modlin I M. Bronchopulmonary neuroendocrine tumors. Cancer 2008; 113(1):5-21.

2. Pelosi G, Rodriguez J, Viale G, Rosai J. Typical and atypical pulmonary carcinoid tumor overdiagnosed as small-cell carcinoma on biopsy specimens: a major pitfall in the management of lung cancer patients. Am. J. Surg. Pathol. 2005; 29(2):179-87.

3. Addis M F, Tanca A, Pagnozzi D, Crobu S, Fanciulli G, Cossu-Rocca P, Uzzau S. Generation of high-quality protein extracts from formalin-fixed, paraffin-embedded tissues. Proteomics. 2009 August; 9(15):3815-23.

4. Zhang, B., Wang, Y., and Su, Y. (2009) Peroxiredoxins, a novel target in cancer radiotherapy. Cancer Lett. 286:154-160.

5. Buxbaum, J. N., and Reixach, N. (2009) Transthyretin: the servant of many masters. Cell. Mol. Life Sci. 66:3095-3101.

6. Zech, V. F. E., Dlaska, M., Tzankov, A., and Hilbe, W. (2006) Prognostic and diagnostic relevance of hnRNP A2/B1, hnRNP B1 and S100 A2 in non-small cell lung cancer. Cancer Detect. Prey. 30:395-402.

7. Chen, G., Wang, H., Gharib, T. G., Huang, C. C., Thomas, D. G., Shedden, K. A., Kuick, R., Taylor, J. M., Kardia, S. L., Misek, D. E., Giordano, T. J., Iannettoni, M. D., Orringer, M. B., Hanash, S. M., and Beer, D. G. (2003) Overexpression of oncoprotein 18 correlates with poor differentiation in lung adenocarcinomas. Mol. Cell. Proteomics 2(2):107-16.

8. Hatakeyama, S., Sugihara, K., Nakayama, J., Akama, T. O., Wong, S. M. A., Kawashima, H., Zhang, J., Smith, D. F., Ohyama, C., Fukuda, M., and Fukuda, M. N. (2009) Identification of mRNA splicing factors as the endothelial receptor for carbohydrate-dependent lung colonization of cancer cells. Proc. Natl. Acad. Sci. U.S.A. 106(9):3095-3100.

9. Huang, D. W., Sherman, B. T., and Lempicki, R. A. (2009) Systematic and integrative analysis of large gene lists using DAVID Bioinformatics Resources. Nat. Protoc. 4(1):44-57.

1. A method for providing a diagnosis for small cell lung carcinoma (SCLC) comprising the steps of:
a. Contacting a biological sample with reagents that allow the extraction of the protein biomarkers selected from the group consisting of: Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1 A chain, Tubulin beta chain;
b. Determining whether the protein biomarkers are differentially expressed in the sample.

2. A method for providing a diagnosis for typical carcinoid tumor (TC) comprising the steps of:
a. Contacting a biological sample with reagents that allow the extraction of the protein biomarkers selected from the group consisting of Abhydrolase domain-containing protein 14B, Annexin AS, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin;
b. Determining whether the protein markers are differentially expressed in the sample.

3. The method according to claim 1, wherein said biological sample comprises a lung cell.

4. The method according to claim 1, wherein said biological samples are fresh or frozen tissue samples.

5. The method according to claim 1, wherein said biological samples are Formalin-fixed, paraffin embedded (FFPE) samples.

6. The method according to claim 1, wherein said differential expression is determined by a method selected from a group consisting of:
a. Detecting mRNA which encodes for the protein biomarker;
b. Detecting the protein encoded by the protein biomarker;
c. Detecting the biological activity of the protein biomarker.

7. A method for providing a diagnosis between small cell lung carcinoma (SCLC) and typical carcinoid tumor (TC) by an antibody-based technique comprising the steps of,
a. Contacting a biological sample with an antibody against one of the following antigens: Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein Al, Putative 40S ribosomal protein S26-like 1, 40S ribosomal protein S4, X isoform, Splicing factor, arginine/serine-rich 9, Stathmin, Tubulin alpha-1 A chain, Tubulin beta chain, Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S 100-A8, Pulmonary surfactant-associated protein A1, Superoxide dismutase [Cu—Zn], Extracellular superoxide dismutase [Cu—Zn], Transgelin-2, Transthyretin;
b. Detecting immunoreactivity.

8. The method according to claim 7, wherein said antibody-based technique is selected from the group consisting of: immunohistochemistry, immunofluorescence and ELISA.

9. Protein biomarker selected from the group consisting of Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1A chain, Tubulin beta chain, Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin as a diagnostic reagent.

10. Use of one or more protein biomarkers selected from group consisting of:
Elongation factor I-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1A chain, Tubulin beta chain as biomarkers for small cell lung carcinoma.

11. Use of one or more protein biomarkers selected from group consisting of: Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin as biomarkers for typical carcinoid tumor.

12. A method of in vitro screening for a compound for treating small cell lung cancer comprising the steps of
a. Contacting a polypeptide which encodes Elongation factor 1-alpha 1, Histone H1.2, Histone H1.5, Histone H2A type 1-D, Histone H3.1t, Histone H4, Heterogeneous nuclear ribonucleoproteins C1/C2, Heterogeneous nuclear ribonucleoprotein K, 60S ribosomal protein L18, 60S ribosomal protein L23a, Heterogeneous nuclear ribonucleoprotein A1, Putative 40S ribosomal protein S26-like 1, 40S ribosomal protein S4, X isoform, Splicing factor, arginine/serine-rich 9, Stathmin, Tubulin alpha-1A chain, Tubulin beta chain, with a test compound;
b. Detecting the biological activity of the polypeptide of step a.

13. A method of in vitro screening for a compound for treating typical carcinoid tumor comprising the steps of
a. Contacting a polypeptide which encodes Abhydrolase domain-containing protein 14B, Annexin A5, Chromogranin-A, Protein CutA, Ferritin heavy chain, Hemoglobin subunit alpha, Hemoglobin subunit beta, Hemoglobin subunit delta, Phosphoglycerate mutase 1, Peroxiredoxin-5, mitochondrial, Peroxiredoxin-6, Pulmonary surfactant-associated protein B, Protein S100-A8, Pulmonary surfactant-associated protein A1, Superoxide dismutase [Cu—Zn], Extracellular superoxide dismutase [Cu—Zn], Transgelin-2, Transthyretin with a test compound;
b. Detecting the biological activity of the polypeptide of step a.

14. The method according to claim 2, wherein said biological sample comprises a lung cell.

15. The method according to claim 2, wherein said biological samples are fresh or frozen tissue samples.

16. The method according to claim 2, wherein said biological samples are Formalin-fixed, paraffin embedded (FFPE) samples.

17. The method according to claim 2, wherein said differential expression is determined by a method selected from a group consisting of
c. Detecting mRNA which encodes for the protein biomarker;
d. Detecting the protein encoded by the protein biomarker;
e. Detecting the biological activity of the protein biomarker.


SEQUENCE LISTING (35 sequences)

SEQ ID NO: 1 - protein, 179 aa, Homo sapiens
MSGGRAPAVLLGGVASLLLSFVWMPALLPVASRLLLLPRVLLTMASGSPPTQPSPASDSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVSDSITVLP

SEQ ID NO: 2 - protein, 154 aa, Homo sapiens [sequence omitted]

SEQ ID NO: 3 - protein, 147 aa, Homo sapiens
MVHLTPEEKTAVNALWGKVNVDAVGGEALGRLLVVYPWTQRFFESFGDLSSPDAVMGNPKVKAHGKKVLGAFSDGLAHLDNLKGTFSQLSELHCDKLHVDPENFRLLGNVLVCVLARNFGKEFTPQMQAAYQKVVAGVANALAHKYH

SEQ ID NO: 4 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 5 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 6 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 7 - protein, 444 aa, Homo sapiens
MREIVHIQAGQCGNQIGAKFWEVISDEHGIDPTGTYHGDSDLQLDRISVYYNEATGGKYVPRAILVDLEPGTMDSVRSGPFGQIFRPDNFVFGQSGAGNNWAKGHYTEGAELVDSVLDVVRKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQVFDAKNMMAACDPRHGRYLTVAAVFRGRMSMKEVDEQMLNVQNKNSSYFVEWIPNNVKTAVCDIPPRGLKMAVTFIGNSTAIQELFKRISEQFTAMFRRKAFLHWYTGEGMDEMEFTEAESNMNDLVSEYQQYQDATAEEEEDFGEEAEEEA

SEQ ID NO: 8 - protein, 306 aa, Homo sapiens
MASNVTNKTDPRSMNSRVFIGNLNTLVVKKSDVEAIFSKYGKIVGCSVHKGFAFVQYVNERNARAAVAGEDGRMIAGQVLDINLAAEPKVNRGKAGVKRSAAEMYGSVTEHPSPSPLLSSSFDLDYDFQRDYYDRMYSYPARVPPPPPIARAVVPSKRQRVSGNTSRRGKSGFNSKSGQRGSSKSGKLKGDDLQAIKKELTQIKQKVDSLLENLEKIEKEQSKQAVEMKNDKSEEEQSSSSVKKDETNVKMESEGGADDSAEEGDLLDDDDNEDRGDDQLELIKDDEKEAEEGEDDRDSANGEDDS

SEQ ID NO: 9 - protein, 381 aa, Homo sapiens
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 10 - protein, 240 aa, Homo sapiens
MLALLCSCLLLAAGASDAWTGEDSAEPNSDSAEWIRDMYAKVTEIWQEVMQRRDDDGALHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVGVCGPGLWERQAREHSERKKRRRESECKAA

SEQ ID NO: 11 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 12 - protein, 372 aa, Homo sapiens
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGGYGGGGPGYSGGSRGYGSGGQGYGNQGSGYGGSGSYDSYNNGGGGGFGGGSGSNFGGGGSYNDFGNYNNQSSNFGPMKGGNFGGRSSGPYGGGGQYFAKPRNQGGYGGSSSSSSYGSGRRF

SEQ ID NO: 13 - protein, 457 aa, Homo sapiens
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 14 - protein, 226 aa, Homo sapiens
MSETAPAETATPAPVEKSPAKKKATKKAAGAGAAKRKATGPPVSELITKAVAASKERNGLSLAALKKALAAGGYDVEKNNSRIKLGLKSLVSKGTLVQTKGTGASGSFKLNKKAASGEAKPKAKKAGAAKAKKPAGATPKKAKKAAGAKKAVKKTPKKAKKPAAAGVKKVAKSPKKAKAAAKPKKATKSPAKPKAVKPKAAKPKAAKPKAAKPKAAKAKKAAAKKK

SEQ ID NO: 15 - protein, 213 aa, Homo sapiens [sequence omitted]

SEQ ID NO: 16 - protein, 149 aa, Homo sapiens
MASSDIQVKELEKRASGQAFELILSPRSKESVPEFPLSPPKKKDLSLEEIQKKLEAAEERRKSHEAEVLKQLAEKREHEKEVLQKAIEENNNFSKMAEEKLTHKMEANKENREAQMAAKLERLREKDKHIEEVRKNKESKDPADETEAD

SEQ ID NO: 17 - protein, 254 aa, Homo sapiens
MAAYKLVLIRHGESAWNLENRFSGWYDADLSPAGHEEAKRGGQALRDAGYEFDICFTSVQKRAIRTLWTVLDAIDQMWLPVVRTWRLNERHYGGLTGLNKAETAAKHGEAQVKIWRRSYDVPPPPMEPDHPFYSNISKDRRYADLTEDQLPSCESLKDTIARALPFWNEEIVPQIKEGKRVLIAAHGNSLRGIVKHLEGLSEEAIMELNLPTGIPIVYELDKNLKPIKPMQFLGDEETVRKAMEAVAAQGKAKK

SEQ ID NO: 18 - protein, 130 aa, Homo sapiens
MSGRGKQGGKARAKAKTRSSRAGLQFPVGRVHRLLRKGNYSERVGAGAPVYLAAVLEYLTAEILELAGNAARDNKKTRIIPRHLQLAIRNDEELNKLLGKVTIAQGGVLPNIQAVLLPKKTESHHKAKGK

SEQ ID NO: 19 - protein, 224 aa, Homo sapiens
MPGGLLLGDVAPNFEANTTVGRIRFHDFLGDSWGILFSHPRDFTPVCTTELGRAAKLAPEFAKRNVKLIALSIDSVEDHLAWSKDINAYNCEEPTEKLPFPIIDDRNRELAILLGMLDPAEKDEKGMPVTARVVFVFGPDKKLKLSILYPATTGRNFDEILRVVISLQLTAEKRVATPVDWKDGDSVMVLPTIPEEEAKKLFPKGVFTKELPSGKKYLRYTPQP

SEQ ID NO: 20 - protein, 214 aa, Homo sapiens
MGLAGVCALRRSAGYILVGGAGGQSAAAAARRCSEGEWASGGVRSFSRAAAAMAPIKVGDAIPAVEVFEGEPGNKVNLAELFKGKKGVLFGVPGAFTPGCSKTHLPGFVEQAEALKAKGVQVVACLSVNDAFVTGEWGRAHKAEGKVRLLADPTGAFGKETDLLLDDSLVSIFGNRRLKRFSMVVQDGIVKALNVEPDGTGLTCSLAPNIISQL

SEQ ID NO: 21 - protein, 199 aa, Homo sapiens
MANRGPAYGLSREVQQKIEKQYDADLEQILIQWITTQCRKDVGRPQPGRENFQNWLKDGTVLCELINALYPEGQAPVKKIQASTMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 22 - protein, 463 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 23 - protein, 263 aa, Homo sapiens [sequence omitted]

SEQ ID NO: 24 - protein, 156 aa, Homo sapiens [sequence omitted]

SEQ ID NO: 25 - protein, 103 aa, Homo sapiens [sequence omitted]

SEQ ID NO: 26 - protein, 462 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 27 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 28 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 29 - protein, 188 aa, Homo sapiens [sequence omitted]

SEQ ID NO: 30 - protein, 221 aa, Homo sapiens
MSGWADERGGEGDGRIYVGNLPTDVREKDLEDLFYKYGRIREIELKNRHGLVPFAFVRFEDPRDAEDAIYGRNGYDYGQCRLRVEFPRTYGGRGGWPRGGRNGPPTRRSDFRVLVSGLPPSGSWQDLKDHMREAGDVCYADVQKDGVGMVEYLRKEDMEYALRKLDDTKFRSHEGETSYIRVYPERSTSYGYSRSRSGSRGRDSPYQSRGSPHYFSPFRPY

SEQ ID NO: 31 - protein, 136 aa, Homo sapiens
MARTKQTARKSTGGKAPRKQLATKVARKSAPATGGVKKPHRYRPGTVALREIRRYQKSTELLIRKLPFQRLMREIAQDFKTDLRFQSSAVMALQEACESYLVGLFEDTNLCVIHAKRVTIMPKDIQLARRIRGERA

SEQ ID NO: 32 - protein, 115 aa, Homo sapiens
MTKKRRNNSHAKKGRGHVQPIRCTNCVRCVPTDKAIKKFVIRNIVEAAAVRDISEVSVFDAYVLPKLYVKLHYCVSCAIHSKVVRNRSREACKDRTPPPRFRPAGAAPRPPPKPM

SEQ ID NO: 33 - protein, 451 aa, Homo sapiens
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGVDSVEGEGEEEGEEY

SEQ ID NO: 34 - protein, 248 aa, Homo sapiens
MWLCPLALNLILMAASGAVCEVKDVCVGSPGIPGTPGSHGLPGRDGRDGLKGDPGPPGPMGPPGEMPCPPGNDGLPGAPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 35 - protein, 210 aa, Homo sapiens
MAASVEQREGTIQVQGQALFFREALPGSGQARFSVLLLHGIRFSSETWQNLGTLHRLAQAGYRAVAIDLPGLGHSKEAAAPAPIGELAPGSFLAAVVDALELGPPVVISPSLSGMYSLPFLTAPGSQLPGFVPVAPICTDKINAANYASVKTPALIVYGDQDPMGQTSFEHLKQLPNHRVLIMKGAGHPCYLDKPEEWHTGLLDFLQGLQ